(19) United States
(12) Patent Application Publication
(10) Pub. No.: US 2025/0281220 A1
(43) Pub. Date: Sep. 11, 2025

(54) Medical Cryotherapy Device with Heat Transfer Body

(71) Applicant: Cryovasc GmbH, Berlin (DE)

(72) Inventors: Wilfried Heiner, Rheinfelden (DE); Karoline Maria Kern, Rheinfelden (DE)

(21) Appl. No.: 18/860,663

(22) PCT Filed: Apr. 27, 2023
(86) PCT No.: PCT/EP2023/061176

§ 371 Date: Oct. 27, 2024

(30) Foreign Application Priority Data
Apr. 27, 2022 (EP) 22170201.2

(51) Int. Cl. A61B 18/02; A61B 17/00; A61B 18/00

(52) U.S. Cl. CPC A61B 18/02; A61B 2017/00867; A61B 2018/0022; A61B 2018/00434; A61B 2018/00577; A61B 2018/00875; A61B 2018/0212

(57) ABSTRACT

The present invention relates to a medical device (1) for cryotherapy, comprising a catheter (3) for cryoablation. The catheter (3) has an at least partly flexible, tubular shaft (5) for introducing the catheter (3) into a bodily vessel, the shaft (5) comprising a first lumen (23) for transporting a coolant to a distal end (11) of the shaft (5) and a second lumen (25) for removing the coolant from the distal end (11) of the shaft (5). The first lumen (23) is connected to a proximal end of the shaft (5) having a coolant source (21). The catheter (3) further comprises an expandable cooling chamber (29). This cooling chamber (29) is arranged at the distal end (11) of the shaft (5) and encloses an end portion of the distal end (11) of the shaft (5) and/or a first lumen portion of the first lumen (23) assigned to an end portion of the distal end (11) of the shaft (5). The coolant is suppliable to the cooling chamber (29) via the first lumen (23) and by means of at least one nozzle (33). The coolant is removable from the cooling channel (29) via the second lumen (25). Thermal energy is transferable via an outer wall (29b) of the expanded cooling chamber (29) such that a cryotherapy is performable at a treatment location. According to the invention, the end portion at the distal end (11) of the shaft (5) comprises a heat transfer element (27) which has an inner element (39) and an outer element (41) enclosing, in particular in the form of a hat, cap or cladding, the inner element (39), wherein the first lumen portion of the first lumen (23), which has a helical or rotationally asymmetric shape, is formed by the interaction of the inner element (39) and the outer element (41). Moreover, the at least one nozzle (33) uses the Joule-Thomson effect. At least one part of the end portion at the distal end (11) of the shaft (5) is in thermally conductive contact with the interior (29a) of the cooling chamber (29). This is done to cool the coolant guided in the first lumen (23) by the coolant supplied to the interior (29a) of the cooling chamber (29).

### CROSS REFERENCE TO RELATED APPLICATIONS

This application is the U.S. national stage of International Application No. PCT/EP2023/061176, filed on 2023 Apr. 27. The international application claims the priority of EP 22170201.2 filed on 2022 Apr. 27; all applications are incorporated by reference herein in their entirety.

### BACKGROUND

The present invention relates to a cryotherapy device, in particular for intravascular cryotherapy, for example for ablation of body tissue, for denervation of perivascular nerves or for stabilization of atherosclerotic plaques.

For example, a cryotherapy device is used to treat high blood pressure and hypertension by denervating the renal perivascular nerves, thereby suppressing or eliminating the activity of these nerves, which is at least partly responsible for high blood pressure. Such a medical device and a method for operating this device is described in the European patent application EP 3 708 100 A1.

This known device shows and describes a medical device for denervation of renal perivascular nerves and has a catheter with a shaft and an inner lumen running in this shaft for supplying a refrigerant. At the distal end of the shaft, a cryoballoon is arranged for cryoablation of the nerves. In the case of its use, that is, during a treatment, the renal artery is touched with the cryoballoon, whereby the intended cryoablation of the nerves is carried out. However, the known device has the disadvantage that the low temperature required for the ablation treatment and the required cooling capacity cannot always be reached, especially not over the desired length of the therapy area.

This disadvantage also occurs in other cryotherapy devices from the prior art, in particular devices dedicated for small blood vessels.

Devices are known from the prior art that also address this problem, such as EP 1 129 669 A1. The device described in this prior art is a cryoablation catheter that comprises a tubular member for supplying a refrigerant. This member is wound around the outer circumference of the distal end of the catheter, which in turn is enclosed by a balloon. The coolant emerging from the tubular member removes heat from the interior of the balloon, so that the balloon can be used for cryotherapy. It is difficult to manufacture a suitably dimensioned catheter according to this state of the art, especially for smaller blood vessels, and its cooling performance would also need to be improved in this case.

### SUMMARY

The present invention relates to a medical device (1) for cryotherapy, comprising a catheter (3) for cryoablation. The catheter (3) has an at least partly flexible, tubular shaft (5) for introducing the catheter (3) into a bodily vessel, the shaft (5) comprising a first lumen (23) for transporting a coolant to a distal end (11) of the shaft (5) and a second lumen (25) for removing the coolant from the distal end (11) of the shaft (5). The first lumen (23) is connected to a proximal end of the shaft (5) having a coolant source (21). The catheter (3) further comprises an expandable cooling chamber (29). This cooling chamber (29) is arranged at the distal end (11) of the shaft (5) and encloses an end portion of the distal end (11) of the shaft (5) and/or a first lumen portion of the first lumen (23) assigned to an end portion of the distal end (11) of the shaft (5). The coolant is suppliable to the cooling chamber (29) via the first lumen (23) and by means of at least one nozzle (33). The coolant is removable from the cooling channel (29) via the second lumen (25). Thermal energy is transferable via an outer wall (29b) of the expanded cooling chamber (29) such that a cryotherapy is performable at a treatment location. According to the invention, the end portion at the distal end (11) of the shaft (5) comprises a heat transfer element (27) which has an inner element (39) and an outer element (41) enclosing, in particular in the form of a hat, cap or cladding, the inner element (39), wherein the first lumen portion of the first lumen (23), which has a helical or rotationally asymmetric shape, is formed by the interaction of the inner element (39) and the outer element (41). Moreover, the at least one nozzle (33) uses the Joule-Thomson effect. At least one part of the end portion at the distal end (11) of the shaft (5) is in thermally conductive contact with the interior (29a) of the cooling chamber (29). This is done to cool the coolant guided in the first lumen (23) by the coolant supplied to the interior (29a) of the cooling chamber (29).

### DETAILED DESCRIPTION

The present invention is therefore based on the task of providing a medical device for cryotherapy, in particular intravascular cryotherapy for the ablation of body tissue, denervation or stabilization of atherosclerotic plaques, which has an improved cooling performance and is cheaper to manufacture. The above task is solved with a medical device according to patent claims 1 and a method according to claim 12.

A medical device for cryotherapy, in particular for intravascular cryotherapy, for the ablation of body tissue, denervation or stabilization of atherosclerotic plaques, comprising a catheter for cryoablation. The catheter has an at least partially flexible, tubular shaft for inserting the catheter into a body vessel, the shaft comprising a first lumen for transporting a refrigerant to a distal end of the shaft and a second lumen for removing the refrigerant from the distal end of the shaft.

The body vessel is, for example, a renal artery of a patient.

The first lumen is connected to a refrigerant source at a proximal end of the shaft. Preferably, the first lumen is an inner lumen and the second lumen is an outer lumen, with the outer lumen in particular surrounding the inner lumen, for example in a concentric arrangement. The catheter further comprises an expandable cooling chamber, in particular a cryoballoon.

This cooling chamber is arranged at the distal end of the shaft and encloses an end portion of the distal end of the shaft and/or a first lumen section of the first lumen, which is associated with an end portion of the distal end of the shaft and which is in particular a distal lumen section of the first lumen. The refrigerant can be supplied to the cooling chamber via the first lumen by means of at least one nozzle or throttle. The cooling chamber can be expanded in particular by supplying the refrigerant, wherein thermal energy can be transferred via an outer wall of the cooling chamber, so that cryotherapy can be carried out at a treatment site. Finally, the refrigerant can be removed from the cooling chamber via the second lumen, with this removal preferably being carried out after the cryotherapy has been performed at the treatment site, for example in the area of the renal perivascular nerves. This cryoablation treatment is carried out by transferring heat energy from the treatment site to the refrigerant via the outer wall of the cooling chamber.

According to the present invention, the at least one nozzle utilizes the Joule-Thomson effect. Furthermore, at least a part of the end portion at the distal end of the shaft is in a thermal-conducting contact with the interior of the cooling chamber for cooling the refrigerant supplied to the interior of the cooling chamber by the refrigerant guided in the first lumen.

The part of the end portion at the distal end of the shaft can be at least a predominant part of the first lumen portion or the entire first lumen portion of the first lumen, which is enclosed by the expandable cooling chamber.

According to the invention, the end portion at the distal end of the shaft comprises a heat transfer body or forms a heat transfer element.

This heat transfer element has an inner member and an outer member surrounding the inner member. The outer member is hat-shaped, cap-shaped or jacket-shaped. The first lumen section of the first lumen is formed by the interaction of the inner member and the outer member. According to the invention, the first lumen section is helical or at least not coaxial but rotationally asymmetrical. Helical also refers to the formation of a double helix or a helix that returns after less than the entire circumference. The goal is to cause a longer path and to control the expansion of the refrigerant.

Preferably, the outer member and/or the inner member is made at least in part of a material having high thermal conductivity. In addition, or alternatively, the inner member may comprise or be a core structure. Such a two-part structure of the heat transfer element comprising an inner and an outer member results in a simple manufacturing method. Preferably, the inner member is fitted or insertable without gap in the outer member.

By providing the at least one nozzle being a Joule-Thomson nozzle and by providing at least a portion of the end portion at the distal end of the shaft being in thermal-conducting contact with the interior of the cooling chamber for cooling the cooling medium conveyed in the first lumen is supplied to the interior of the cooling chamber, a medical device is created with enhanced cooling performance, which can be used to carry out an even more efficient denervation treatment, in particular of renal perivascular nerves.

Preferably, the refrigerant supplied from the refrigerant source to the first lumen is gaseous and is liquefied by cooling the refrigerant in the first lumen due to the transfer of thermal energy between the first lumen section of the first lumen and the interior of the cooling chamber. The refrigerant is preferably gaseous until it enters the cooling chamber area, and is converted or can be converted into the liquid state with the help of the heat transfer element by means of the cooling. The cooling or the transfer of thermal energy takes place in particular in the first lumen section of the first lumen.

### Nitrous Oxide (N)

A particularly advantageous embodiment of the invention provides that the first lumen section of the first lumen has an enlarged, preferably substantially enlarged, boundary or surface area for transferring the thermal energy from the interior of the cooling chamber to the first lumen in comparison to a second lumen section, which is arranged outside the cooling chamber, wherein this second lumen section has the same extent in the axial direction of the first lumen as the first lumen section in the longitudinal direction. As a result of this increased boundary or surface area, the axial flow velocity, in particular an axial component of the refrigerant flow velocity, of the refrigerant in the region of the first lumen section of the first lumen can be reduced compared to the axial flow velocity in the second lumen section. Transfer of thermal energy from the first lumen particularly effects transfer to and absorption by the refrigerant present in the cooling chamber at that time. In particular, a heat transfer arrangement is proposed by this invention that can lead to a further increase in cooling efficiency.

A preferred embodiment of the helical shape provides that the first lumen, in particular the refrigerant in the first lumen, in the first lumen section after a single complete revolution around the core structure, passes through a section in the axial direction that is no greater than twice the diameter of the first lumen. Preferably, this section is slightly greater than the single diameter of the first lumen. In a cross-sectional view, this section corresponds to the distance between the centers of a first cross-section and a second cross-section of the first lumen, with a single complete revolution of the first lumen around the core structure between the first and second cross-sections. This particularly tightly wound helix arrangement results in a particular increase in surface area, which in turn results in a particularly effective heat transfer surface.

An advantageous embodiment of the invention is characterized in that the first lumen section of the first lumen is represented as a groove on the surface of the inner member. This surface of the inner member may be formed as a cylindrical surface. Preferably, this groove is represented as a helical or spiral groove. In addition, the recess can have a U-shaped cross section. Such a structure is helpful for a particularly simple production of the heat transfer element, preferably with particularly small dimensions. Such a minimized structure of the catheter is favorable for the further use of a gentle treatment method.

According to a further embodiment, it may be provided that the first lumen section of the first lumen is formed by a core structure inserted into the first lumen at the distal end, with a particularly preferred helical recess.

Accordingly, the heat transfer element is formed by a core or core element inserted into the first lumen together with the peripheral wall of a tubular member which defines the first lumen per se. The at least one groove, which is preferably present in the core structure, together with the inner surface of the tubular member, which surrounds the core structure in a peripheral manner, defines the first lumen section of the first lumen. The initially gaseous refrigerant flows through this and is cooled and preferentially liquefied from the outside, through the wall of the tubular member, in order to evaporate at the end of the core member via a nozzle, which is preferably formed between the core member and the tubular member, and to effect cooling of the cooling chamber.

According to a particularly preferred embodiment, the depth of the groove decreases in the direction of the nozzle. This increases the velocity and reduces the static pressure. However, a constant or widening cross-section of the groove in the direction of the nozzle may also be provided. These variations apply to all embodiments of the invention.

In a particular embodiment, which can also be flexibly combined, the first lumen section of the first lumen can also be designed as a nozzle overall, e.g. as a Venturi nozzle. In this case, in the transition region between the first lumen and the first lumen section, there is a narrowing of the cross-section down to a minimum cross-section, at which the groove has the smallest cross-section. Subsequently, the cross-section of the groove widens until it exits the nozzle into the cooling chamber. It may be provided that an opening is provided in the area of the smallest cross-section for sucking in evaporated refrigerant from the cooling chamber.

It may be provided that a lower pressure than the fluid pressure in the first lumen can be set in the cooling chamber, in particular in the cryo-balloon, by the refrigerant exiting the nozzle. This pressure is advantageously lower than a maximum pressure associated or associable with the strength of the outer wall of the cooling chamber. In this way, overloading of the outer wall with the risk of it breaking can be avoided. The nozzle is particularly advantageous if it is a venturi nozzle, which can lead to an increase in the cooling effect due to the resulting increased expansion of the refrigerant when it enters the cooling chamber. According to one embodiment of the medical device, the outer wall of the cooling chamber, in particular the envelope of the cryo-balloon, is formed from a thin-walled, stretchable material and is designed to be double-walled. In order to increase safety during treatment, in particular to avoid an embolism during treatment due to a defect in the outer wall of the cooling chamber and a cooling medium entering the patient's artery, at least one sensor for detecting the condition of this wall can be included. This can be realized, for example, by a sensor that monitors at least one parameter in the space defined by the inner and outer walls of the wall. Advantageously, it is provided that when such a defect is detected by the sensor, at least one warning signal is output by a control unit of the medical device, and, preferably, the source of the refrigerant is automatically switched off so that no further refrigerant can be introduced into the cooling chamber.

Alternatively or in addition to the double-walled design, the outer wall of the cooling chamber, in particular the envelope of the cryo-balloon, can be formed from a material comprising a shape memory alloy and/or can be at least partially surrounded by a basket, at least when the medical device is in use. This basket can also be formed from a material that comprises or is a shape memory alloy, for example Nitinol. With the help of this basket enveloping the outer wall, a maximum expansion of the cooling chamber can be defined.

Preferably, the medical device, in particular the catheter, comprises at least one pressure sensor for determining the blood pressure. Such blood pressure measurement, preferably during the treatment, can enable constant control of the treatment success, in particular in the case of an execution of several successive cryoablation treatments, so that the treatment can be terminated when a desired result occurs. The medical device can additionally or alternatively comprise at least one electrode for delivering a pulse to stimulate the renal perivascular nerves, so that it can be determined whether a corresponding stimulation results in an increase in blood pressure. If such an increase is detected, in particular to an unacceptable extent, the cryoablation treatment is started or continued or repeated until there is no further increase in blood pressure or only an acceptable increase in blood pressure after renewed stimulation.

Another embodiment of the invention provides that the catheter comprises measuring electrodes arranged on the outside of the cooling chamber for a resistance or impedance measurement to detect the state of a denervation treatment. In this way, a value for the lesion depth can be determined. If the determined value exceeds a predetermined limit, the treatment is interrupted to prevent excessive tissue damage that could be harmful to the patient's health. The electrodes for stimulating the renal perivascular nerves are also used as measuring electrodes for impedance measurement, so that no additional electrodes have to be attached.

The above task is further solved with a method for operating a medical device for denervation according to claim 12. The method according to the invention for operating a medical device is intended for cryotherapy. This medical device, which is particularly designed according to one of the aforementioned embodiments, comprises a catheter for cryoablation, which has an at least partially flexible, tubular shaft for introducing the catheter into a body, for example into a renal artery of a patient, and an expandable cooling chamber, in particular a cryo-balloon, which is arranged at the distal end of the shaft and encloses an end portion of the distal end. The refrigerant is transported in a first lumen from a refrigerant source from the proximal end of the shaft to the distal end of the shaft. Subsequently, the refrigerant is supplied to the expandable cooling chamber by means of at least one nozzle located at the end or end region of the first lumen. The refrigerant is then removed from the distal end of the shaft through a second lumen. Thermal energy is transferred across an outer wall of the cooling chamber to perform cryotherapy at the treatment site. According to the invention, the refrigerant is expanded and thereby cooled as a result of an isenthalpic pressure reduction according to the Joule-Thomson effect when it enters the interior of the cooling chamber. In this process, the expanded refrigerant in the interior of the cooling chamber cools the refrigerant that is located in an end portion of the distal end of the first lumen at the same time through the surface of this end portion according to the reverse flow principle via a heat transfer element. Furthermore, it is envisaged that the refrigerant preferably changes its aggregate state twice on its way from the entry point in the proximal end of the first lumen to the interior of the cooling chamber. The method according to the invention is particularly designed in such a way that the refrigerant is supplied in the gaseous state to a proximal end of the first lumen and is passed on in this gaseous state to the end portion, surrounded by the cooling chamber, of the distal end of the first lumen, is cooled in the end portion of the distal end of the first lumen, as a result of the transfer of thermal energy, to at least one transition into the liquid state, and exits through the at least one nozzle from the first lumen and enters the interior of the cooling chamber, a further cooling of the refrigerant takes place due to the Joule-Thomson effect, and inside the cooling chamber is used both to cool the refrigerant in the end portion of the distal end of the first lumen and for cryoablation via the outer wall of the cooling chamber.

The individual procedural steps described above are preferably carried out in exactly this order.

Preferably, the refrigerant within the first lumen is cooled to less than 15° C., preferably less than 10° C., at a pressure built up in the first lumen in the range between 0.04 to 0.06 hPa, preferably at least approximately 0.05 hPa.

### BRIEF DESCRIPTION OF THE DRAWINGS

It should be noted that all features of the medical device that are described by way of device features and that can be carried out in the device should also be considered to be disclosed as process features. The invention will be explained in more detail below, using the drawings.

FIG. 1 shows a schematic representation of a cryotherapy device with a catheter;

FIG. 2 shows an enlarged detailed view according to Detail II of FIG. 1 of the distal end of the catheter in a longitudinal section in a first embodiment;

FIG. 3 shows the distal end according to FIG. 2 with its component parts in a 3D exploded view;

FIG. 4A) shows an enlarged detailed view according to detail II in FIG. 1 of the distal end of the catheter in a longitudinal section according to a second embodiment; and

FIG. 4B) shows another enlarged detailed view according to detail II in FIG. 1 of the distal end of the catheter in a longitudinal section according to a second embodiment.

### DETAILED DESCRIPTION OF THE PREFERRED EMBODIMENTS

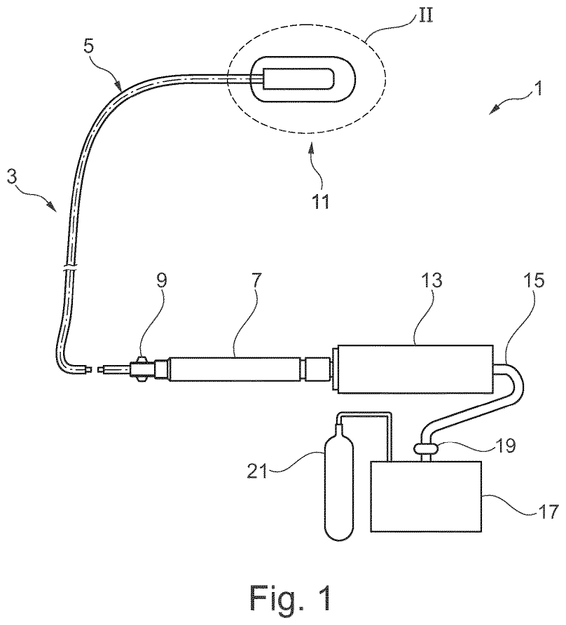

FIG. 1 shows an embodiment of a cryotherapy device 1 according to the present invention. The cryotherapy device 1 comprises a catheter 3 having a shaft 5, a handling grip 7 and a control button 9. The control button 9 is movable in the axial direction relative to the handling grip 7, in such a way that the distal end 11 of the catheter 3 can be directed for the purpose of controlling the direction during insertion of the catheter. FIG. 1 shows an optional heat exchanger device 13, which in this case is coupled to the handling grip 7. By means of this heat exchanger device 13, which is connected to a control unit 17 via a connecting hose 15, it is possible to precool a cooling medium before it enters shaft 5. A control valve 19 is arranged in the connecting hose and serves to regulate the gas flow, which serves as a refrigerant and flows from a gas cylinder 21.

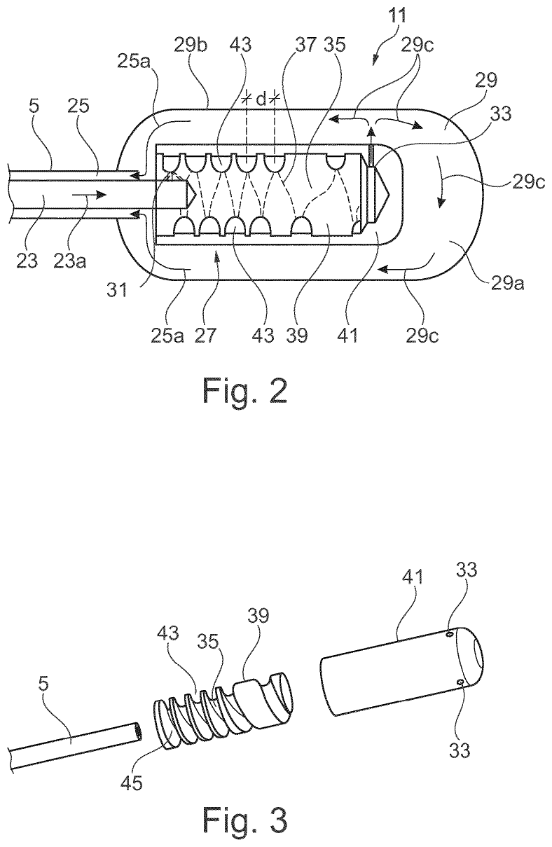

Nitrous oxide (N2O) is preferably used as the refrigerant Control valve 19 serves to regulate the gas flow through shaft 5 to the distal catheter end 11, which also represents the actual treatment unit for cold or cryoablation. As can be seen in particular from the longitudinal section in the enlarged detailed view in FIG. 2, shaft 5 has a first, in the present case inner, lumen 23 for supplying 23a and transporting the refrigerant to the distal catheter end 11. This inner lumen 23 extends from the refrigerant source 21, or in the present example from the heat exchanger device 13, to the distal catheter end 11. From the distal catheter end 11, the refrigerant “used up” by the treatment, which is described in more detail below, is conveyed through a second lumen 25, which is also arranged in the shaft 5 and is in the present case an outer lumen 25, to the outlet 25a, wherein the outlet 25a usually leads into the anaesthetic gas suction system of the treatment room. Alternatively, the refrigerant can also be recirculated, so that instead of the gas cylinder 21, a gas reservoir with a return for refrigerant from the outer lumen 25 is also conceivable.

The enlarged longitudinal section view according to FIG. 2 illustrates further details of the distal catheter end 11, which generally comprises a heat transfer element 27 and an expandable cryo-balloon 29 enclosing this heat transfer element. The heat transfer element 27 essentially conducts the refrigerant from an input side located on a first side of the heat transfer element 27 to an output side located on a second side of the heat transfer element 27, which is opposite the first side. For this purpose, a channel inlet 31 is arranged on the input side, into which the part of the inner lumen 23 guided in the shaft 5 opens. After flowing through the heat transfer element 27, the refrigerant passes through nozzle 33 and enters the interior 29a of the cryo-balloon 29, which is bounded by a balloon envelope/wall 29b. The entry of the gaseous refrigerant into the interior 29a of the cryo-balloon 29 causes the latter to expand and the balloon envelope 29b to stretch accordingly. Inside 29a of the cryo-balloon 29, the refrigerant is distributed as indicated at 29c and flows back to the first side of the heat transfer element 27, which is closest to the shaft 5, so that the refrigerant is discharged through the outer lumen 25 as indicated at 25a. On its way through the interior of the cryo-balloon 29, the refrigerant cools the cryo-balloon 29, so that a corresponding temperature is also present at the balloon envelope/wall 29b, which, due to its thinness, is particularly suitable for heat transfer. During treatment, the balloon envelope 29b then touches, for example, the renal perivascular nerves to be discharged, the position of which was controlled with catheter 3 via the respective renal artery.

In the longitudinal section of FIG. 2, it is visible which path the refrigerant takes through the heat transfer element 27. After entering the heat transfer element 27 at the channel inlet 31, the inner lumen 23 passes close to an interface with the interior 29a of the cryo-balloon 29 along a helical path that is guided around a core structure 35, as indicated in FIG. 2 at 37 as auxiliary lines.

The 3D exploded view in FIG. 3 shows how the cylindrical heat transfer element 27 is manufactured. This consists essentially of two members, an inner member 39 and an outer member 41. In the inner member 39, which is also cylindrical, the inner lumen 23 is introduced as a helical structure, in particular during a production step by means of a milling process or with the aid of a laser beam, on the surface of the jacket, whereby the path that the refrigerant takes through the heat transfer element 27 is defined. According to a particularly cost-effective variant, the core element can also be manufactured by injection molding. As shown in FIGS. 2 and 3, the introduced helical structure is shown as a helical groove 43 with a U-shaped cross section. The pitch of the helical structure, i.e. the distance d between the centers of a first sectional area and a second sectional area of the inner lumen 23, wherein between the first and second sectional area there is a single complete revolution of the first lumen 23 around the core structure 35, is set in such a way that a particularly large boundary or surface area is set up between the inner lumen 23 and the interior 29a of the cryo-balloon 29 for the purpose of a particularly large heat transfer.

Preferably, the pitch of the helical structure is not constant. Preferably, the helical structure has a larger pitch in the direction of its distal end. The increase in the pitch of the helical structure of the lumen from proximal to distal results in a reduction in pressure in the inner lumen 23. It may also be envisaged that the pitch of the helical structure initially reduces from proximal to distal and only after reaching a minimum pitch does it increase again towards the distal end.

It has been shown that the refrigerant is first compressed when it enters the helical structure. This liquefies the refrigerant or keeps it liquid. As the pitch of the helical structure increases towards the distal end, the refrigerant expands again. This causes at least part of the refrigerant to change phase preferentially from liquid to vapor. It has been shown that this design influences the cooling capacity (it increases). The same applies if the cross-section of the helical or rotationally asymmetrical lumen increases in the direction of the distal end.

After the helical structure has been introduced on the surface of the inner member 39, the heat transfer element 27 is completed by placing the cylindrical, cap-shaped outer member 41 over the inner member 39, in particular by press-fitting it. The outer member 41 encloses the inner member 39 with a precise fit, i.e. without play between these two members 39, 41. In this way, the helical inner lumen 23 in the heat transfer element 27 is also defined.

In FIG. 3, the inlet opening into the helical inner lumen 23 is still indicated near the channel inlet 31 at 45. Thus, after channel inlet 31, the inner lumen 23 is deflected by about 90°, so that the refrigerant enters the helix structure in a radial direction. In addition, the design according to FIG. 3 provides several nozzles 33, which contribute to a more uniform entry of refrigerant into the interior 29a of the cryo-balloon 29 and also to a further increase in cooling efficiency, since a corresponding amount of cold is generated at each individual nozzle 33.

The illustration in FIG. 2 also shows the mode of operation of the cryo-balloon 29 and the heat transfer element 27 enclosed by it. The refrigerant is introduced in the gaseous state from the gas cylinder 21 as the refrigerant source via the inner lumen 23 into the heat transfer element 27 and passes through the helical inner lumen 23 arranged therein until it exits into the interior 29a of the cryo-balloon 29. Due to the Joule-Thomson effect, the refrigerant is expanded when it exits the nozzle 33 and thus cooled, so that the temperature of the refrigerant inside 29a of the cryoballoon 29, i.e. in the groove 43, is lower than the temperature of the refrigerant in the helical first lumen 23 at the same time. Due to this temperature difference between the interior 29a of the cryo-balloon 29 and the inner lumen 23, an exchange of heat energy takes place at their boundary or surface, so that the temperature of the refrigerant in the inner lumen 23 is further reduced. To increase the heat transfer, it is also possible, according to an embodiment not shown here, to provide that the surface of the heat transfer element 27 facing the balloon is increased by projections, ribs and the like compared to a cylindrical surface.

As the refrigerant is continuously discharged from the interior 29a of the cryoballoon 29 via the outer lumen 25, with new refrigerant, which has been further reduced in temperature, being supplied from the inner lumen 23 in the helix structure, a further temperature reduction successively occurs in the interior 29a of the cryoballoon 29. As a result of the further heat exchange across the boundary or surface between the interior 29a of the cryo-balloon 29 and the inner lumen 23 in the heat transfer element 27, a temperature value is reached at which the refrigerant in the inner lumen 23 in the heat transfer element 27 liquefies. When this liquefied refrigerant is sprayed through the nozzle 33 into the interior 29a of the cryo-balloon 29, it returns to its gaseous state due to the resulting expansion, causing the refrigerant to cool down which is based both on the Joule-Thomson effect and on the phase transition from liquid to gas, by which heat energy is removed from the environment of the nozzle 33, thus leading to a further cooling of the refrigerant. In this way, the cooling effect in the described system is further increased so that a sufficient amount of cold is available for the cryoablation treatment. If the cross-section of the groove changes along the flow path within the first section of the lumen and in particular in the heat transfer element, the transition from liquid to gas may also have occurred before the nozzle exits into the cooling chamber. This can also or additionally be achieved by increasing the pitch. Surprisingly, it has been found that this further increases the cooling capacity.

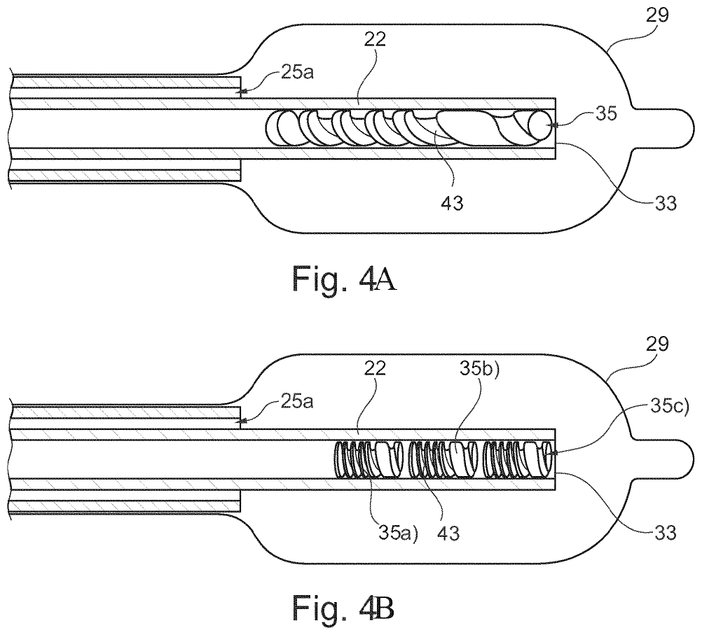

The representation according to FIG. 4a) shows the production of the cylindrically shaped heat transfer element 27 according to a second embodiment.

Here, the heat transfer element is essentially formed by two members, an inner member and an outer member. The inner member is formed as a core 35 having a helically extending groove 43. During the manufacture of the device, this core is pushed with a snug fit into the inner lumen 23 of the catheter and, if necessary, glued. The inner lumen is formed by the wall of tubular member 22. The wall of tubular member 22, together with the core 35, forms the heat transfer element 27.

The structure of the core can correspond to the structure of the core shown in FIG. 3. In this design, the first lumen section of the first lumen is formed by the groove 43 of the core 35 and the tubular member 22 surrounding the core 35.

Here, it may be provided that the depth of the groove changes. It may be provided that the cross-section of the groove widens from a point of minimal cross-section towards the nozzle outlet. In the area of the point with the minimum cross-section, according to a further embodiment, one or more recesses may also be provided in the wall of the tubular member, so that refrigerant could be drawn from the cooling chamber by the vacuum created by the Venturi effect and expelled into the cooling chamber at the end of the nozzle.

FIG. 4b) shows a modification of the embodiment shown in FIG. 4a). Here, the heat transfer element 27 is formed by the wall of the tubular element 22 together with the core 35. In contrast to FIG. 4a), the core is formed from a plurality of segments. This makes the catheter as a whole less stiff and thus better able to reach the treatment site. Finally, it should be noted that in FIGS. 1 to 4, the details of the cryotherapy device 1 are only shown schematically in some parts. In particular, only the features and components necessary for understanding the invention are illustrated in the representation according to FIG. 2. For the practical use of the catheter, further elements are required or helpful. In particular, the provision of a J-wire for handling the catheter 3 during insertion into the patient's body, i.e. the introduction, for example, into the renal artery, as shown, for example, in FIG. 2 of EP 3 708 100 A1 with reference sign 7, is helpful for the practical design of the catheter. Furthermore, elements and components useful for successful treatment, such as the generally mentioned pressure sensors and electrodes for stimulating nerve cells or measuring electrodes, are not shown in the illustrations, but should also be considered as disclosed in the context of the invention.

### LIST OF REFERENCE NUMERALS

1 cryotherapy device
3 catheter
5 shaft
7 handle
9 control button
11 distal end
13 heat exchanger device
15 connecting tube
17 control unit
19 control valve
21 gas cylinder
22 tubular member
23 inner lumen
23a refrigerant supply
25 outer lumen
25a refrigerant discharge
27 heat transfer element
29 cryo-balloon
29a balloon interior
29b balloon envelope/wall
29c flow in interior of balloon
31 channel inlet
33 nozzle
35 core structure
37 helix
39 inner member
41 outer member
43 groove
45 inlet
d distance

1. A medical device for cryotherapy comprising a catheter, said catheter comprising:
1.1 an at least partially flexible, tubular shaft for inserting the catheter into a body vessel, the shaft comprising a first, preferably inner, lumen for transporting a refrigerant to a distal end of the shaft and a second, preferably outer lumen surrounding the inner lumen for removing the refrigerant from the distal end of the shaft, the first lumen being connected to a source of refrigerant at a proximal end of the shaft,
1.2 an expandable cooling chamber, in particular a cryo-balloon, which is arranged at the distal end of the shaft and encloses an end portion of the distal end of the shaft and/or a first lumen section, associated with an end portion of the distal end of the shaft, of the first lumen, to which the refrigerant can be supplied via the first lumen by means of at least one nozzle, and from which the refrigerant can be removed via the second lumen, wherein the cooling chamber can be expanded, in particular by supplying the refrigerant, and wherein thermal energy can be transferred via an outer wall of the cooling chamber, so that cryotherapy can be carried out at a treatment site
characterized in that the end portion at the distal end of the shaft comprises a heat transfer element or forms a heat transfer element which has an inner member, comprising or being designed as a core structure,
and an outer member enclosing the inner member, in particular in the form of a cap, a hat or a jacket, the first lumen portion of the first lumen being formed by the interaction of the first lumen section of the first lumen formed by the interaction of the inner member and the outer member, the first lumen section of the first lumen being formed in a helical or rotationally asymmetrical manner and the at least one nozzle being a nozzle utilizing the Joule-Thomson effect and at least part of the end portion at the distal end of the shaft, in particular at least a predominant part of the first lumen section of the first lumen enclosed by the expandable cooling chamber, is in heat-conducting contact with the interior of the cooling chamber for cooling the refrigerant conveyed in the first lumen by the refrigerant supplied to the interior of the cooling chamber.

2. The medical device according to claim 1, characterized in that the refrigerant supplied from the refrigerant source to the first lumen is at least partially gaseous and in that, due to the cooling and/or compression of the refrigerant in the first lumen, in particular in the first/distal lumen section of the first lumen, as a result of the transfer of thermal energy between the interior of the cooling chamber and the first lumen, in particular the first lumen section of the first lumen, the refrigerant, which is at least partially gaseous, can be converted into the liquid state, in particular when it enters the cooling chamber region.

3. The medical device according to claim 1, characterized in that the pitch of the helix and/or the cross section of the helical lumen increases in the direction of the distal end.

4. The medical device according to claim 1, characterized in that the helix shape is configured in such a way that the first lumen, in particular the refrigerant in the first lumen, in the first lumen section after a single complete revolution around the core structure, passes through a section in the axial direction which is no greater than twice the diameter, preferably slightly greater than the single diameter, of the first lumen.

5. The medical device according to claim 1, characterized in that the first lumen section of the first lumen is formed as a recess on the surface, in particular formed as a cylinder jacket surface, of the inner member, wherein the recess is preferably formed as a helical or spiral groove and/or with a U-shaped cross section.

6. The medical device according to claim 1, characterized in that the first lumen section of the first lumen is formed by a core structure having a helical or rotationally asymmetric recess, which is inserted into the first lumen at the distal end, together with the tubular member surrounding the first lumen.

7. The medical device according to claim 1, characterized in that, in the cooling chamber, in particular in the cryo-balloon, the outlet of the refrigerant from the nozzle allows a pressure lower than the fluid pressure in the first lumen can be set, which is preferably lower than a maximum pressure associated or associable with the strength of the outer wall of the cooling chamber, the nozzle being in particular a Venturi nozzle.

8. The medical apparatus according to claim 1, characterized in that the outer wall of the cooling chamber, in particular the envelope of the cryo-balloon, is formed from a thin-walled stretchable material and is designed to be double-walled, at least one sensor for detecting the state of the outer wall being included in particular, for example for monitoring a parameter in the intermediate space defined by the inner and outer walls of the envelope.

9. The medical device according to claim 1, characterized in that the outer wall of the cooling chamber, in particular the envelope of the cryo-balloon, at least when using the medical device, is formed from a material comprising a shape memory alloy, and/or is at least partially surrounded by a cage, in particular a cage formed from a material comprising a shape memory alloy.

10. The medical device according to claim 1, characterized in that the medical device, in particular the catheter, comprises at least one pressure sensor for determining the blood pressure, in particular during the treatment, and/or at least one electrode for emitting an electrical impulse for stimulating nerves.

11. The medical device according to claim 1, characterized in that the medical device, in particular the catheter, comprises measuring electrodes, preferably arranged on the outside of the cooling chamber, for a electrical resistance measurement for detecting the state of a denervation treatment.

12. A method for operating a medical device for denervation, in particular of renal perivascular nerves, the device comprising a catheter for cryoablation, which has an at least partially flexible, tubular shaft for introducing the catheter into a body vessel, in particular into a renal artery of a patient, and an expandable cooling chamber, in particular a cryoballoon, arranged at the distal end of the shaft and enclosing an end portion of the distal end, in particular a cryoballoon, wherein the refrigerant is transported in a first lumen from a refrigerant source from the proximal end of the shaft to the distal end of the shaft, is supplied to the expandable cooling chamber by means of at least one nozzle arranged at the end or an end region of the first lumen, and is removed through a second lumen from the distal end of the shaft, wherein thermal energy is transferred via an outer wall of the cooling chamber for carrying out cryotherapy at a treatment site, and wherein the medical device is configured in particular according claim 1, characterized in that the refrigerant expands and is thereby cooled as a result of an isenthalpic pressure reduction according to the Joule-Thomson effect upon entering the interior of the cooling chamber, wherein the expanded refrigerant in the interior of the cooling chamber cools the refrigerant present at the same time in an end portion of the distal end of the first lumen through the surface of this end portion according to the counterflow principle, and wherein the refrigerant changes its state of aggregation preferably twice on its way from the inlet to the proximal end of the first lumen to the interior of the cooling chamber.

13. The method according to claim 12, characterized in that the refrigerant is supplied in the gaseous state to a proximal end of the first lumen and is passed in this gaseous state to the end portion of the distal end of the first lumen enclosed by the cooling chamber, is cooled in the end portion of the distal end of the first lumen is cooled to at least a transition to the liquid state as a result of thermal energy transfer, emerges from the first lumen through the at least one nozzle and enters the interior of the cooling chamber, wherein, upon entry into the interior of the cooling chamber, a further refrigerant due to the Joule-Thomson effect, and is used in the interior of the cooling chamber both for cooling the refrigerant in the end portion of the distal end of the first lumen and for cryoablation via the outer wall of the cooling chamber.

14. The method according to claim 12, characterized in that the refrigerant within the first lumen is cooled to less than 15° C., preferably less than 10° C., at a pressure built up in the first lumen in the range between 0.04 to 0.06 hPa, preferably at least approximately 0.05 hPa.
